(19) United States
(12) Patent Application Publication
(10) Pub. No.: US 2011/0150976 A1
(43) Pub. Date: Jun. 23, 2011

(54) TRANSDERMAL DELIVERY OF OLIGOSACCHARIDES

(75) Inventors: Galit Levin, Nordiya (IL); Hagit Sacks, Modi'in (IL); Meir Stern, Rehovot (IL); Galit Mazooz, Rehovot (IL)

(73) Assignee: TRANSPHARMA MEDICAL LTD.

(21) Appl. No.: 13/061,466

(22) PCT Filed: Sep. 10, 2009
(86) PCT No.: PCT/IL09/00889

§ 371 Date: Feb. 28, 2011

Related U.S. Application Data
(60) Provisional application No. 61/095,637, filed on Sep. 10, 2008.

(51) Int. Cl. A61K 31/727; A61K 31/715; A61K 9/70; A61P 7/02; A61M 37/00

(52) U.S. Cl. 424/449; 514/54; 514/56; 604/20

(57) ABSTRACT

The present invention relates to a system for facilitating transdermal delivery of oligosaccharides, The system includes an apparatus that generates micro-channels in the skin of a subject and a skin patch. The patch includes a pharmaceutical composition containing an active agent of an oligosaccharide. Particularly, the oligosaccharide has 5 to 20 monosaccharide units, such as a pentasaccharide, which exerts anticoagulant activity. The system is capable of delivering the oligosaccharides into the blood circulation for treating thromboembolic diseases.

### FIELD OF THE INVENTION

The present invention relates to a system for facilitating transdermal delivery of oligosaccharides comprising an apparatus that generates micro-channels in the skin of a subject and a skin patch comprising a pharmaceutical composition comprising as an active agent an oligosaccharide. Particularly, the composition comprises an oligosaccharide of 5-20 monosaccharide units, such as a pentasaccharide, which exerts anticoagulant activity. The system is capable of delivering the oligosaccharides into the blood circulation for treating thromboembolic diseases.

### BACKGROUND OF THE INVENTION

Blood clotting is a complex physiological phenomenon. Certain stimuli, such as contact activation and tissue factor, trigger the subsequent activation of a series of clotting factors present in blood plasma. Irrespective of the nature of the stimulus, the final steps are identical, activated factor X (Xa) activates factor II (also known as prothrombin), which, in its activated form (factor IIa, also known as thrombin), gives rise to partial proteolysis of soluble fibrinogen with release of insoluble fibrin, which is one of the main constituents of a blood clot.

Under normal physiological conditions, the activity of the clotting factors is regulated by proteins such as antithrombin-III (AT-III) and heparin cofactor II (HC II), which are also present in plasma. AT-III exerts inhibitory activity on a certain number of clotting factors, and in particular on factors Xa and thrombin.

Inhibition of factor Xa or of thrombin is thus the preferred means for obtaining anticoagulant and antithrombotic activity, since these two factors are involved in the final two steps of clotting, which are independent of the triggering stimulus.

Heparin is a polysaccharide of the glycosaminoglycan family which is a commonly used anticoagulant obtained from biological sources such as intestinal mucosa. In the presence of heparin, thrombin undergoes accelerated inactivation by AT-III which involves conformational changes in both heparin and AT-III.

The structural features of heparin that are required for the interaction with AT-III have been resolved. Studies of fragmented heparin resulted in the identification of a pentasaccharide fragment accounting for the minimal high-affinity structure that binds to AT-III. In this high-affinity fragment eight sulfate groups are present: four of the sulfate groups were found to be essential for binding to AT-III, whereas the other groups were found to attribute to higher affinity. Synthesis of oligosaccharides, particularly those described in European Patent Nos. 84999; 529715 and 621282, revealed that the synthetic oligosaccharides selectively inhibit factor Xa via AT-III. These synthetic oligosaccharides which correspond to the anti-thrombin-binding domain (ABD) of heparin manifest anti-thrombotic activity in venous thrombosis.

Further synthesis of pentasaccharides yielded many analogs, among which the more stable and active pentasaccharide analogs fondaparinux and idraparinux. Fondaparinux produced by GlaxoSmithKline and registered in the U.S. and Europe under the name of Arixtra®, is currently used for treating patient undergoing abdominal surgery who are at risk of thromboembolic complications. The half-life of fondaparinux in human is approximately 17 hours, which makes once-daily administration by subcutaneous injection possible. Idraparinux, produced by Sanofi-Synthelabo and Organon, displays higher activity and a longer duration of action than fondaparinux and was shown to be effective in the treatment of venous thromboembolic events when administered at a dose of 2.5 mg once weekly.

U.S. Pat. No. 4,841,041 discloses pentasaccharides having anti-thrombotic activity and especially anti factor Xa activity. U.S. Pat. No. 4,841,041 further discloses that the pentasaccharides can be administered enterally and parenterally, wherein parenteral administration is carried out by subcutaneous, intramuscular or intravenous injection.

U.S. Pat. No. 5,378,829 discloses sulfated glycosaminoglycanoid derivatives of heparin and heparin sulfate type, including pentasaccharides, and uses thereof for the treatment of venous thrombosis and for the inhibition of smooth muscle cell proliferation. According to U.S. Pat. No. 5,378,829, the compounds may be administered enterally or parenterally, by injection or inhalation.

U.S. Pat. No. 6,174,863 discloses pentasaccharides having (1-4C)alkoxy or sulfate groups that replace hydroxyl groups in the ATIII binding domain of heparin, the total number of the sulfate groups is 4 to 6. The pentasaccharides according to U.S. Pat. No. 6,174,863 may be administered enterally or parenterally, by injection or inhalation.

U.S. Pat. No. 6,541,488 discloses uses of direct or indirect selective inhibitors of factor Xa acting via antithrombin III in combination with a compound having anti-platelet activity for preventing and treating thromboembolic arterial diseases.

U.S. Pat. No. 6,670,338 discloses pentasaccharides in which one of the O-alkyl groups is replaced with an alkylene bridge, thus producing a pentasaccharide lacking conformational flexibility but gaining advantageous biological properties. According to U.S. Pat. No. 6,670,338, the pentasaccharides can be administered orally, sublingually, subcutaneously, intramuscularly, intravenously, transdermally, transmucosally, locally or rectally, though subcutaneous administration is indicated to be the preferred route of administration.

U.S. Pat. No. 6,844,329 discloses conjugates of synthetic pentasaccharides exerting antithrombotic activity and having a covalent bond with biotin or a derivative thereof, the antithrombotic activity can be neutralized by avidin that interacts with biotin.

European Patent No. 1446131 discloses the use of specific dose of fondaparinux sodium for the treatment of Acute Coronary Syndromes (ACS).

International Application Publication No. WO 03/022860 discloses preparation of synthetic monosaccharides, disaccharides, trisaccharides, tetrasaccharides and pentasaccharides, including fondaparinux, for use in the preparation of synthetic heparinoids.

International Application Publication No. WO 2006/019894 discloses methods for treating bronchial disorders by inhalation of one or more compounds that act by inhibiting thrombin directly and indirectly. Among the compounds, fondaparinux and pentasaccharides related to fondaparinux are specifically disclosed.

International Application Publication No. WO 2006/082184 discloses conjugates of polypeptides and oligosaccharides, the oligosaccharides comprise 4-18 monosaccharide units and have affinity to antithrombin III.

Petitou et al. (Nature 398: 417-422, 1999) described the synthesis of oligosaccharides of 16-, 18-, 19-, and 20-mer saccharide units which contain the anti-thrombin binding domain (A-domain) and the thrombin binding domain (T-domain). These oligosaccharides, specifically the 19- and 20-mer oligosaccharides, were found to be highly active in inhibiting both Xa and thrombin activity, their activity was as potent as that of standard heparin (Petitou et al., ibid).

International Application Publication No. WO 2004/039428 assigned to the applicant of the present application discloses a printed patch which comprises a dried pharmaceutical composition comprising an active agent, a system for facilitating transdermal delivery of an active agent comprising an apparatus that generates micro-channels in the skin of a subject and said printed patch, and methods of use thereof. The active agent according to WO 2004/039428 is preferably a hydrophilic active agent and can be a polypeptide or a polynucleotide.

International Application Publication No. WO 2004/112689 assigned to the applicant of the present invention discloses a system for intradermal or transdermal delivery of a water-soluble, poorly water-soluble or water insoluble cosmetic agent comprising an apparatus that generates micro-channels in the skin of a subject and a cosmetic or dermatological composition comprising a water-soluble, poorly water-soluble, or water-insoluble cosmetic agent. WO 2004/112689 further discloses uses of the system for intradermal or transdermal delivery of a cosmetic agent for treating skin conditions including acne, cellulite, skin wrinkles, and hyperpigmentation, among others.

There is an unmet need for improved methods for delivery of oligosaccharides into the blood circulation to achieve a therapeutic effect.

### SUMMARY OF THE INVENTION

The present invention provides a system for facilitating transdermal delivery of an oligosaccharide, the system comprising an apparatus that generates micro-channels in an area of the skin of a subject and a skin patch comprising a pharmaceutical composition comprising as an active agent an oligosaccharide. The present invention further provides uses of the system for preventing or treating venous and arterial thromboembolic diseases.

It is now disclosed for the first time that generating micro-channels in an area of the skin of a subject and affixing a skin patch comprising a pharmaceutical composition which comprises as an active agent a sulfated oligosaccharide to the area of the skin where micro-channels have been generated, resulted in transdermal delivery of high amounts of the oligosaccharide into the blood circulation.

The present invention further discloses that affixing a skin patch comprising a pharmaceutical composition formulated as a viscous liquid comprising a sulfated oligosaccharide as the active agent and a water-soluble thickening agent to an area of the skin of a subject where micro-channels have been generated, resulted in a prolonged increase in the plasma level of the oligosaccharide, thereby achieving a long-lasting therapeutic effect attributed to the oligosaccharide delivered.

The present invention discloses unexpectedly that transdermal delivery of a sulfated oligosaccharide having anti-coagulant activity through the generated micro-channels enables achieving a therapeutic effect without causing hemorrhage.

The methods of the present invention are virtually painless, achieve highly efficient delivery of the oligosaccharide over a prolonged period of time, and avoid the expected adverse side-effect, e.g., hemorrhage. Thus, the methods of the present invention are preferable over the commonly used painful and difficult subcutaneous injections of the sulfated pentasaccharide fondaparinux sodium.

The principles of the present invention are exemplified herein below using the sulfated pentasaccharide fondaparinux. It is explicitly intended that the system and methods of the present invention are applicable to any oligosaccharide, including a sulfated oligosaccharide having anti-coagulant activity, particularly an oligosaccharide of 5-20 monosaccharide units which exerts anti-coagulant activity.

According to a first aspect, the present invention provides a system for facilitating transdermal delivery of an oligosaccharide through skin of a subject comprising: an apparatus capable of generating a plurality of micro-channels in an area of the skin of the subject, and a skin patch comprising a pharmaceutical composition comprising as an active agent an oligosaccharide and a pharmaceutically acceptable carrier.

According to some embodiments, the oligosaccharide has anti-coagulant activity. According to additional embodiments, the oligosaccharide having anti-coagulant activity consists of 5 to 20 monosaccharide units comprising, in anionic form, at least the general formula I:

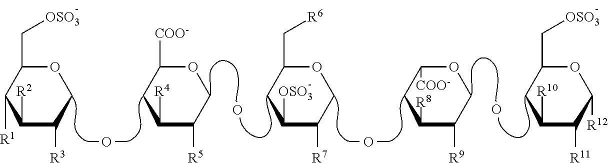

wherein:

R₁ and R₁₂ each is independently OH, (C₁-C₄) alkoxy, or an oligosaccharide consisting of 1 to 8 monosaccharide units,

R₂, R₄, R₅, and R₈ each is independently OH, (C₁-C₄) alkoxy, or OSO₃⁻,

R₃, R₇, and R₁₁ each is independently NHSO₃ (C₁-C₄) alkoxy, OH, or OSO₃⁻,

R₆ and R₉ each is independently OSO₃⁻, or (C₁-C₄) alkoxy, and

R₁₀ is OH, (C₁-C₄) alkoxy, OSO₃ or COO⁻;

or a pharmaceutically acceptable salt thereof.

According to further embodiments, the oligosaccharide is a pentasaccharide, alternatively the oligosaccharide consists of 6 monosaccharide units, 7, 8, 9, 10, 11, 12, 13, 14, 15, 16, 16, 18, 19, or of 20 monosaccharide units. According to additional embodiments, the oligosaccharide comprises, in anionic form, at least two repeats of formula I. According to further embodiments, the oligosaccharide comprises three repeats or 4 repeats of formula I.

According to a certain embodiment, the pentasaccharide is fondaparinux or a pharmaceutically acceptable salt thereof.

According to another embodiment, the pentasaccharide is idraparinux or a pharmaceutically acceptable salt thereof.

According to another embodiment, the pharmaceutically acceptable salt is a sodium salt. According to an exemplary embodiment, the pentasaccharide is fondaparinux sodium.

According to some embodiments, the pharmaceutical composition within the skin patch is formulated as a viscous liquid, liquid, or in a dry form.

According to additional embodiments, if the pharmaceutical composition is formulated as a viscous liquid, said pharmaceutical composition further comprises a water-soluble thickening agent. According to further embodiments, the water-soluble thickening agent is selected from the group consisting of water-soluble cellulose derivatives, polypropylene oxide, polyethylene oxide, polyoxyethylene-polyoxypropylene copolymers, polyvinylalcohol, polyethylene glycol, and combinations thereof. According to still further embodiments, the water-soluble cellulose derivative is selected from the group consisting of a hydroxyalkyl cellulose, alkyl cellulose, and alkylhydroxyalkyl cellulose, e.g., hydroxyethyl cellulose, hydroxypropyl cellulose, methylcellulose, hydroxypropyl methylcellulose, and the like. According to a certain embodiment, the water soluble cellulose derivative is hydroxyethyl cellulose.

According to some embodiments, the water-soluble thickening agent such as a water-soluble cellulose derivative is present in an amount ranging from about 0.5% to about 3.5% (w/w) of the composition, preferably from about 1% to about 3% (w/w) of the composition, and more preferably from about 1% to about 2% (w/w) of the composition. According to a certain embodiment, the water-soluble thickening agent is hydroxyethyl cellulose present in the composition in an amount of about 1.0% (w/w) of the formulation.

According to additional embodiments, the viscous liquid has a viscosity of at least about 300 centipoise (cps), alternatively of at least about 1000 cps, further alternatively of at least about 3000 cps, 5000 cps, 10000 cps, 20000 cps, or yet further alternatively of up to about 40000 cps. According to a certain embodiment, the viscous liquid has a viscosity of about 300 cps to about 3000 cps.

According to an exemplary embodiment, the pharmaceutical composition is formulated as a viscous liquid comprising as an active agent fondaparinux sodium, hydroxyethyl cellulose as a water-soluble cellulose derivative, and water as the pharmaceutically acceptable carrier, the viscosity of the composition ranges from about 300 cps to 3000 cps.

According to further embodiments, the pharmaceutical composition further comprises an agent selected from the group consisting of a buffering agent, a stabilizer and an anti-oxidant.

According to further embodiments, the patch further comprises at least one of the following layers: a backing layer, an adhesive, a rate-controlling layer, and a release liner.

According to some embodiments, the apparatus comprises:
(a) an electrode cartridge comprising a plurality of electrodes; and
(b) a main unit comprising a control unit, which is adapted to apply electrical energy of radio frequency to the electrodes when the electrodes are in vicinity of or in contact with the stratum corneum of the skin, enabling ablation of the stratum corneum in an area beneath the electrodes, thereby generating the plurality of micro-channels.

According to additional embodiments, the electrode cartridge is adapted to generate the plurality of micro-channels having uniform shape and dimensions.

According to another aspect, the present invention provides a method for treating a subject suffering from a thromboembolic disease comprising:
(a) generating a plurality of micro-channels in an area of the skin of the subject;
(b) affixing a skin patch to the area of skin in which the plurality of micro-channels is present, the patch comprising a pharmaceutical composition comprising a therapeutically effective amount of an oligosaccharide having anti-coagulant activity and a pharmaceutically acceptable carrier; and
(c) delivering the oligosaccharide into the blood circulation, thereby treating the subject from said disease.

According to yet further aspect, the present invention provides a method for transdermal delivery of an oligosaccharide comprising the steps of:
(a) generating a plurality of micro-channels in an area of the skin of a subject in need of such treatment;
(b) affixing a skin patch to the area of skin in which the plurality of micro-channels is present, the patch comprises a pharmaceutical composition comprising a therapeutically effective amount of an oligosaccharide and a pharmaceutically acceptable carrier; and
(c) delivering the oligosaccharide into the blood circulation.

According to some embodiments, the oligosaccharide to be transdermally delivered by the methods of the present invention is an oligosaccharide having anti-coagulant activity. According to additional embodiments, the oligosaccharide having anti-coagulant activity to be transdermally delivered by the methods of the present invention comprising, in anionic form, at least the general formula I. According to further embodiments, the oligosaccharide of formula I consists of 5 monosaccharide units. Alternatively, the oligosaccharide consists of 6, 7, 8, 9, 10, 11, 12, 13, 14, 15, 16, 17, 18, 19 or 20 monosaccharide units. According to a certain embodiment, the oligosaccharide is fondaparinux or a pharmaceutically acceptable salt thereof. According to an exemplary embodiment, the oligosaccharide is fondaparinux sodium. According to another embodiment, the oligosaccharide to be transdermally delivered by the methods of the present invention is idraparinux or a pharmaceutically acceptable salt thereof.

According to some embodiments, the oligosaccharide is delivered into the blood circulation in an amount of at least about 1 mg, alternatively in an amount of at least about 2 mg, 3 mg, or at least about 5 mg.

According to some embodiments, the step of generating the plurality of micro-channels in an area of the skin of a subject is performed by an apparatus which comprises:
(a) an electrode cartridge comprising a plurality of electrodes; and
(b) a main unit comprising a control unit, which is adapted to apply electrical energy of radio frequency to the electrodes when the electrodes are in vicinity of or in contact with the stratum corneum of the skin, enabling ablation of the stratum corneum in an area beneath the electrodes, thereby generating the plurality of micro-channels.

According to one embodiment, the electrode cartridge to be used for the methods of the present invention is adapted to generate the plurality of micro-channels having uniform shape and dimensions.

According to some embodiments, the thromboembolic disease is a venous thromboembolic disease or an arterial thromboembolic disease selected from the group consisting of deep vein thrombosis, pulmonary embolism, thrombophlebitis, arterial occlusion from thrombosis or embolism, arterial reocclusion during or after angioplasty or thrombolysis, restenosis following arterial injury or invasive cardiological procedures, postoperative venous thrombosis or embolism, acute or chronic atherosclerosis, stroke, and myocardial infarction.

According to yet further aspect, the present invention provides use of a system which comprises an apparatus capable of generating a plurality of micro-channels in an area of the skin of a subject, and a skin patch comprising a pharmaceutical composition comprising as an active agent an oligosaccharide and a pharmaceutically acceptable carrier for transdermal delivery of the oligosaccharide. According to some embodiments, the oligosaccharide to be used in the system of the present invention has anti-coagulant activity. According to additional embodiments, the oligosaccharide having anti-coagulant activity to be used comprising, in anionic form, at least the general formula I.

According to another aspect, the present invention provides a skin patch comprising a pharmaceutical composition comprising as an active agent an oligosaccharide of 5 to 20 monosaccharide units, the oligosaccharide having anti-coagulant activity comprising, in anionic form, at least the general formula I:

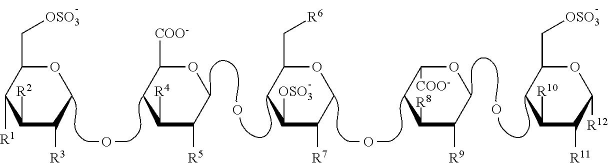

wherein:

R₁ and R₁₂ each is independently OH, (C₁-C₄) alkoxy, or an oligosaccharide consisting of 1 to 8 monosaccharide units,

R₂, R₄, R₅, and R₈ each is independently OH, (C₁-C₄) alkoxy, or OSO₃⁻,

R₃, R₇, and R₁₁ each is independently NHSO₃⁻, (C₁-C₄) alkoxy, OH, or OSO₃⁻,

R₆ and R₉ each is independently OSO₃⁻, or (C₁-C₄) alkoxy, and

R₁₀ is OH, (C₁-C₄) alkoxy, OSO₃⁻, or COO⁻;

or a pharmaceutically acceptable salt thereof, further comprising a pharmaceutically acceptable carrier.

According to some embodiments, the oligosaccharide is a pentasaccharide, alternatively the oligosaccharide consists of 6 monosaccharide units, 7, 8, 9, 10, 11, 12, 13, 14, 15, 16, 16, 18, 19, or of 20 monosaccharide units. According to additional embodiments, the oligosaccharide comprises, in anionic form, at least two repeats of formula I. According to further embodiments, the oligosaccharide comprises three repeats or 4 repeats of formula I.

According to a certain embodiment, the pentasaccharide is fondaparinux or a pharmaceutically acceptable salt thereof.

According to another embodiment, the pentasaccharide is idraparinux or a pharmaceutically acceptable salt thereof.

According to certain embodiments, the pharmaceutically acceptable salt is a sodium salt. According to an exemplary embodiment, the pentasaccharide is fondaparinux sodium.

According to some embodiments, the pharmaceutical composition within the skin patch is formulated as a viscous liquid, liquid, or in a dry form.

According to additional embodiments, if the pharmaceutical composition is formulated as a viscous liquid, said pharmaceutical composition further comprises a water-soluble thickening agent. According to further embodiments, the water-soluble thickening agent is selected from the group consisting of water-soluble cellulose derivatives, polypropylene oxide, polyethylene oxide, polyoxyethylene-polyoxypropylene copolymers, polyvinylalcohol, polyethylene glycol, and combinations thereof. According to still further embodiments, the water-soluble cellulose derivative is selected from the group consisting of a hydroxyalkyl cellulose, alkyl cellulose, and alkylhydroxyalkyl cellulose, e.g., hydroxyethyl cellulose, hydroxypropyl cellulose, methylcellulose, hydroxypropyl methylcellulose, and the like. According to a certain embodiment, the water-soluble cellulose derivative is hydroxyethyl cellulose.

According to some embodiments, the water-soluble thickening agent such as a water-soluble cellulose derivative is present in an amount ranging from about 0.5% to about 3.5% (w/w) of the composition, preferably from about 1% to about 3% (w/w) of the composition, and more preferably from about 1% to about 2% (w/w) of the composition. According to a certain embodiment, hydroxyethyl cellulose is present in the composition in an amount of about 1.0% (w/w) of the formulation.

According to additional embodiments, the viscous liquid having a viscosity of at least about 300 centipoise (cps), alternatively of at least about 1000 cps, further alternatively of at least about 3000 cps, 5000 cps, 10000 cps, 20000 cps, or yet further alternatively of up to about 40000 cps. According to a certain embodiment, the viscous liquid has a viscosity of about 300 cps to about 3000 cps.

According to a certain embodiment, the skin patch comprising a pharmaceutical composition which is formulated as a viscous liquid comprising as an active agent fondaparinux sodium, hydroxyethyl cellulose as a water-soluble cellulose derivative, and water as the pharmaceutically acceptable carrier, the viscosity of the composition ranges from about 300 cps to 3000 cps

According to further embodiments, the pharmaceutical composition further comprises an agent selected from the group consisting of a buffering agent, a stabilizer and an anti-oxidant.

According to further embodiments, the patch further comprises at least one of the following layers: a backing layer, an adhesive, a rate-controlling layer, and a release liner.

These and other embodiments of the present invention will be better understood in relation to the figures, description, examples and claims that follow.

### BRIEF DESCRIPTION OF THE FIGURES

FIG. 1 is a graph showing plasma levels of fondaparinux in pigs. The animals were treated with ViaDerm to generate micro-channels at a density of 150 or 300 micro-channels/cm² of skin and then a printed patch containing 5 mg fondaparinux was affixed to the treated skin. As a control, pigs were injected subcutaneously with 1 mg of fondaparinux.

FIG. 2 is a graph showing plasma levels of fondaparinux in pigs. The animals were treated at two sites with ViaDerm to generate 300 micro-channels/cm² of skin and then a silicone pouch containing 100 mg/ml fondaparinux solution was affixed to each treated site (total dose 28 mg/pig). As a control, pigs were injected subcutaneously with 1 mg of fondaparinux.

FIG. 3 is a graph showing plasma levels of fondaparinux in pigs. The animals were treated with ViaDerm to generate 300 micro-channels/cm² of skin and then a silicone pouch, each containing 200 mg/ml (total dose 14 mg/pig) fondaparinux in hydroxyethyl cellulose (HEC) aqueous gel at the indicated viscosity, was affixed to the treated skin. As a control, pigs were injected subcutaneously with 2.5 mg of fondaparinux.

FIGS. 4A-B show micrographs of skin sections. Pigs were treated with ViaDerm to generate micro-channels at a density of 450 micro-channels/cm² of skin and then a skin patch comprising dried fondaparinux was applied to the treated skin. Skin samples were removed 13 hours after treatment, fixed and stained with hematoxylin and eosin. FIG. 4A, magnification×100; FIG. 4B, magnification×400.

### DETAILED DESCRIPTION OF THE INVENTION

The present invention provides a system for facilitating transdermal delivery of an oligosaccharide, the system comprises an apparatus that generates micro-channels in an area of the skin of a subject and a skin patch comprising a pharmaceutical composition which comprises as an active agent an oligosaccharide, particularly an oligosaccharide having anti-coagulant activity, and more particularly an oligosaccharide of 5-20 monosaccharide units of general formula I. The present invention further provides uses of said system for preventing or treating venous and arterial thromboembolic diseases.

The term “micro-channel” as used in the context of the present specification and claims refers to a hydrophilic pathway generally extending from the surface of the skin through all or a significant part of the stratum corneum and may reach into the epidermis or dermis, through which molecules can diffuse. It should be appreciated that after micro channels have been generated in the stratum corneum, the apparatus is removed from the skin, and the active agent is delivered from a patch subsequently placed on the skin into the systemic circulation by diffusion only.

The present invention incorporates devices and techniques for creating micro-channels by inducing ablation of the stratum corneum by electric current or spark generation at radio frequency (RF), including the apparatus referred to as ViaDerm or MicroDerm, as disclosed in one or more of the following: U.S. Pat. Nos. 6,148,232; 5,983,135; 6,597,946; 6,611,706; 6,708,060; WO 2004/039428; Sintov et al., J. Controlled Release 89: 311-320, 2003; the content of which is incorporated by reference as if fully set forth herein.

The present invention provides a skin patch comprising a pharmaceutical composition comprising as an active agent an oligosaccharide, particularly an oligosaccharide having anti-coagulant activity and a pharmaceutically acceptable carrier. The terms “patch” and “skin patch” refer to a patch to be affixed to the skin of a subject and are used interchangeably throughout the specification and claims.

According to some embodiments, the oligosaccharide of the present invention consists of 5 to 20 monosaccharide units as set forth in general formula I:

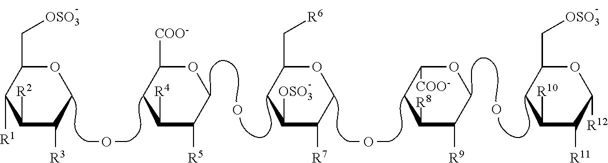

wherein:

R₁ and R₁₂ each is independently OH, (C₁-C₄) alkoxy, or an oligosaccharide consisting of 1 to 8 monosaccharide units,

R₂, R₄, R₅, and R₈ each is independently OH, (C₁-C₄) alkoxy, or OSO₃⁻,

R₃, R₇, and R₁₁ each is independently NHSO₃ (C₁-C₄) alkoxy, OH, or OSO₃⁻,

R₆ and R₉ each is independently OSO₃⁻, or (C₁-C₄) alkoxy, and

R₁₀ is OH, (C₁-C₄) alkoxy, OSO₃⁻, or COO⁻;

or a pharmaceutically acceptable salt thereof.

Preferred oligosaccharides are pentasaccharides selected from the group consisting of fondaparinux and idraparinux.

Fondaparinux is, in its anionic form, of the formula I wherein:

R₁, R₂, R₄, R₅, R₈, and R₁₀ each is independently OH;

R₃, R₇, and R₁₁ each is independently NHSO₃⁻;

R₆ and R₉ each is independently OSO₃⁻; and

R₁₂ is C₁ alkoxy (see, for example, Petitou et al., Angew. Chem. Int. Ed. 43: 3118-3133, 2004, the content of which is incorporated by reference as if set forth herein).

Idraparinux is, in its anionic form, of the formula I wherein:

R₁, R₂, R₃, R₄, R₅, R₈, R₉, and R₁₂ each is independently C₁ alkoxy, and

R₆, R₇, R₁₀ and R₁₁ each is independently OSO₃⁻.

It is to be understood that the present invention excludes polysaccharides or mucopolysaccharides. The present invention encompasses oligosaccharides having up to 50 monosaccharide units. Preferably, the oligosaccharides have affinity to AT-III, inhibit factor Xa activity and hence having anti-coagulant activity.

The oligosaccharides of the invention are natural occurring oligosaccharides or synthetic oligosaccharides that can be prepared by any known methods of sugar chemistry, and in particular by reacting a monosaccharide containing protective groups such as described by T. W. Green, in Protective Groups in Organic Synthesis (Wiley, N.Y. 1981), on the hydroxyl radicals and optionally on the carboxyl radicals, if present, with another protected monosaccharide, to form a disaccharide which is then reacted with another protected monosaccharide to form a protected trisaccharide, from which a protected tetrasaccharide and then a protected pentasaccharide and so on can be obtained. The protected oligosaccharides are then deprotected and modified by for example, sulfation or alkylation, or partially deprotected, then modified in order to obtain the compounds of the invention (see, for example WO 03/022860 which discloses methods for the preparation of mono-, di-, tri-, tetra- and pentasaccharides, and Petitou et al., ibid., the content of which is incorporated as if fully set forth herein).

The oligosaccharides of the present invention can be present in acidic form or in the form of a pharmaceutically acceptable salt. In the acidic form, the —COO, NHSO₃ and —SO₃ functions are in the form —COOH, NHSO₃H and —SO₃H, respectively.

The term “pharmaceutically acceptable salt” of the oligosaccharides of the invention is intended to refer to oligosaccharides in which one or more of the —COO and/or —SO₃ and/or NHSO₃ functions are ionically bonded to a pharmaceutically acceptable cation. Any inorganic or organic base which gives pharmaceutically acceptable salts can be used. Sodium hydroxide, potassium hydroxide, calcium hydroxide or magnesium hydroxide is preferably used. The sodium salts of the oligosaccharides of the invention are the preferred salts.

The present invention encompasses conjugates that comprise the oligosaccharides of the invention. Thus, an oligosaccharide of the invention can be conjugated to a peptide or polypeptide (see, for example, WO 2006/082184, the content of which is incorporated by reference as if fully set forth herein). Alternatively, the oligosaccharide of the invention can be coupled to a biotin molecule (see, for example, U.S. Pat. No. 6,844,329, the content of which is incorporated by reference as if fully set forth herein).

The pharmaceutical composition within the skin patch can be formulated in a form of a viscous liquid, liquid, or in a dry form.

The term “viscous liquid” refers to a solution having a viscosity higher than the viscosity of the pharmaceutically acceptable carrier, e.g., water or a buffer. The viscous liquid according to the present invention has a viscosity of at least about 300 centipoise (cps). When formulated as a viscous liquid, the pharmaceutical composition comprises a water-soluble thickening agent.

Thickening agents are typically added to liquid compositions to increase the viscosity of the resulting composition. A composition having an increased viscosity is beneficial for topical applications where controlled release and/or avoiding run-off are important. The thickening agent according to the present invention should raise the viscosity of the composition to at least about 300 centipoise (cps), alternatively to at least about 1000 cps, 2000 cps, 3000 cps, 4000 cps, 5000 cps, 10000 cps, 15000 cps, 20000 cps, 30000 cps, or further alternatively to up to about 40000 cps. Preferably, the thickening agent of the present invention raises the viscosity of the composition to about 300 cps to about 3,000 cps. Viscosity is measured using a rotating spindle viscometer.

Various thickening agents can be used to hold or retain the pharmaceutical composition and include, but not limited to, biopolymers and hydrophilic synthetic polymers.

The biopolymers and derivatives thereof, which can be used according to the invention include, but are not limited to, water-soluble cellulose derivatives such as, for example, hydroxyethyl cellulose, hydroxypropyl cellulose, methyl cellulose, and carboxymethyl cellulose, chitin, carboxymethylchitin, chitosan, alginates, gelatin, dextran, alginic acid, galactomannan, gum arabic, tragacanth gum, gelan gum, karaya gum, agar, xanthan gum, curdlan, pullulan, starch, glucomannan, xyloglucan, lentinan, glycosaminoglycans such as hyaluronan (see, for example, U.S. Pat. Nos. 5,418,222; 5,510,418; 5,512,301; 5,681,568; 6,596,293; 6,565,879 and references therein and Curr. Pharm. Biotechnol., 2003, 4(5): 283-302; Crit. Rev. Ther. Drug Carrier Syst., 2001, 18(5): 459-501; Eur. J. Pharm. Sci., 2001, 14(3): 201-7; Adv. Drug Deliv. Rev., 2001, 51 (1-3): 81-96; and Int. J. Pharm., 2001, 221(1-2): 1-22).

Hydrophilic synthetic polymers that can be used according to the invention include biodegradable and non-degradable polymers including, but not limited to, polypropylene oxide, polyethylene oxide, polyoxyethylene-polyoxypropylene copolymers, polyvinylalcohol, polyethylene glycol, polyacrylate, polyurethanes, and other hydrophilic synthetic polymers known in the art. It should be appreciated to one skilled in the art that chemical conjugates whereby biopolymers are conjugated with hydrophilic synthetic polymers to form the drug reservoir layer are also encompassed in the present invention.

The term “water soluble” thickening agent or cellulose derivative as used herein refers to a thickening agent or cellulose derivative that typically has solubility in water at a concentration of up to about 1 gr/12 ml at room temperature.

Typically, the skin patch comprises a drug reservoir layer which comprises the pharmaceutical composition. The drug reservoir layer of the present invention is typically thin, flexible, and conformable to provide intimate contact with a body skin, and is able to release the oligosaccharide from the reservoir at rates sufficient to achieve therapeutically effective transdermal fluxes of the oligosaccharide.

According to some embodiments, the skin patch can comprise the pharmaceutical composition in a dry form, i.e., dried or lyophilized pharmaceutical composition.

The term “dried or lyophilized pharmaceutical composition” as used in the context of the present specification and claims refers to a pharmaceutical composition of which the residual moisture is below about 20%, preferably below about 10%, more preferably below about 5%, and most preferably below about 3% of the final composition's weight.

After application of a skin patch comprising a dried or lyophilized pharmaceutical composition on the pretreated new skin environment, the pharmaceutical composition is dissolved in fluid that comes out of the micro-channels, and is then absorbed through the micro-channels into the body. This approach is particularly suitable for drugs that do not irritate the skin even at high concentrations.

According to certain embodiments of the present invention, it is possible to monitor and to obtain a relative evaluation of the loss of fluids that come out from the micro-channels in the new skin environment with respect to the loss of fluids that come out from the skin prior to ablation of the stratum corneum. This type of measurement is also termed herein “transepidermal water loss” or “TEWL”, and is described in the foregoing examples.

Thus, a patch comprising a pharmaceutical composition in the solid state may have several advantages:
i. relatively high delivery rates, due to the delivery from a saturated solution or suspension;
ii. may enable production of thin and convenient patch, instead of reservoir patches;
iii. practical, as it enables usage of very small amounts of expensive agents.

Methods for preparing different types of dry patches, specifically methods that are suitable for accurately placing small amounts of an active agent as a dry agent onto a solid support from which they will be released are disclosed in WO 04/039428 assigned to the applicant of the present application, the content of which is incorporated by reference as if fully set forth herein) herein.

According to an exemplary embodiment, a skin patch comprising a dried pharmaceutical composition comprising as an active agent the oligosaccharide of the present invention is a printed patch.

Printing methods encompass techniques in which small droplets of a solution or suspension of a pharmaceutical composition are placed on a uniform liner in a controlled manner. The droplets dry rapidly and leave solid dots of the pharmaceutical composition. The dose is accurately determined by the concentration of the active agent in the solution or suspension and the configuration and programming of the manufacturing instrument. Besides the therapeutically active agent, the pharmaceutical composition may advantageously include other materials, such as solubility increasing agents, stabilizers, and polymers.

In order to penetrate into the skin and the blood circulation, the pharmaceutical composition within the printed dots on the liner is dissolved in the fluids that are exuded from the skin through micro-channels.

Methods known in the art for applying droplets include a small volume (one to several microliter) syringe or an array of syringes, a combination of a small volume syringe or an array of syringes with a metering pump, an array of small pins, tips of the pins dipped in the solution/suspension, printing with a device like an ink jet printer, printing with a cartridge containing the solution of the pharmaceutical composition, spraying of a thin film of a solution of active drug on a liner and the like.

To enable adhesion of the printed patch to the new environment skin the printing is prepared on a transdermal adhesive backing liner. Alternatively, a suitable adhesive can be printed between the prints of the drug, on a non-adherent liner.

Drying can be carried under controlled conditions for example by changing the temperature, humidity or pressure.

According to a certain embodiment, the printed patch which comprises the dried pharmaceutical composition comprising the oligosaccharide of the present invention is a printed patch wherein the pharmaceutical composition is present on a non-adhesive liner which is made of a material that is not permeable to the oligosaccharide (see WO 04/039428).

The patch of the present invention can comprise one or more rate controlling layers, which are usually microporous membranes. Rate controlling layers comprise biopolymers and/or synthetic polymers. The rate controlling layers are devoid of an active agent. Representative materials useful for forming rate-controlling layers include, but are not limited to, polyolefins such as polyethylene and polypropylene, polyamides, polyesters, ethylene-ethacrylate copolymer, ethylene-vinyl acetate copolymer, ethylene-vinyl methylacetate copolymer, ethylene-vinyl ethylacetate copolymer, ethylene-vinyl propylacetate copolymer, polyisoprene, polyacrylonitrile, ethylene-propylene copolymer, cellulose acetate and cellulose nitrate, polytetrafluoroethylene (“Teflon”), polycarbonate, polyvinylidene difluoride (PVDF), polysulfones, and the like.

The patch can further comprise an adhesive layer. Alternatively or additionally, the drug reservoir layer can itself have adhesive properties. The patch can further comprise a backing layer.

Typically, a backing layer functions as the primary structural element of a skin patch and provides flexibility and, preferably, occlusivity. The material used for the backing layer should be inert and incapable of absorbing an active agent or any component of a pharmaceutical composition. The backing layer preferably comprises a flexible and/or elastomeric material that serves as a protective covering to prevent loss of the active agent via transmission through the upper surface of the patch, and will preferably impart a degree of occlusivity to the patch, such that the area of the body surface covered by the patch becomes hydrated during use. The backing layer also prevents dehydration of the pharmaceutical composition. The material used for the backing layer should permit the patch to follow the contours of the skin and be worn comfortably on areas of skin such as at joints or other points of flexure, that are normally subjected to mechanical strain with little or no likelihood of the patch disengaging from the skin due to differences in the flexibility or resiliency of the skin and the patch. Examples of materials useful for the backing layer are non-adhesive materials such as polyesters, polyolefins including monolayers or coextruded multilayers, polyethylene, polypropylene, vinyliden chloride/vinyl chloride copolymer, ethylene/vinyl acetate copolymer, polyurethanes, polyether amides, and the like. The occlusive backing layer may be covered by an adhesive layer to allow sticking the patch on to the skin in a way that does not interfere with drug delivery to the micro-channels treated skin.

During storage and prior to use, the patch can include a release liner. Immediately prior to use, this liner is removed so that the patch may be affixed to the skin. The release liner should be made from a drug or active agent impermeable material, and is a disposable element, which serves only to protect the patch prior to application.

According to the principles of the invention, the pharmaceutical composition comprises a pharmaceutically acceptable carrier.

The term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, excipient, or vehicle with which the active agent is administered. Carriers are more or less inert substances when added to a pharmaceutical composition to confer suitable consistency or form to the composition.

As used herein a “pharmaceutically acceptable carrier” is aqueous solutions or suspensions. Examples of aqueous carriers include water, saline and buffered media, alcoholic/aqueous solutions, or suspensions.

To optimize desirable characteristics of a pharmaceutical composition, various additives can be optionally included in the pharmaceutical composition. Thus, to improve the stability of the active agent, a suitable stabilizing agent can be added. Suitable stabilizing agents include, but are not limited to, most sugars, preferably trehalose, mannitol, lactose, sucrose, and glucose. In order to improve water absorption, hygroscopic additives may be added as well. To produce a pH that is compatible with a particular active agent being used, a suitable buffer can be used. Suitable buffers include most of the commonly known and utilized biological buffers, including acetate, citrate, phosphate buffer, or succinate buffer. A compatible pH is one that maintains the stability of an active agent, optimizes its therapeutic effect or protects against its degradation. A suitable pH is generally from about 3 to about 8, preferably from about 4 to about 7. Additionally, anti-oxidants (e.g., ascorbic acid, sodium metabisulfite) or preservatives (e.g., Thimerosal, benzyl alcohol, parabens, m-cresol) can be added as well.

### Devices for Enhancing Transdermal Delivery of Oligosaccharides

The system of the present invention comprises an apparatus for enhancing transdermal delivery of an oligosaccharide. According to the invention the apparatus is used to generate a new skin environment through which an oligosaccharide is delivered efficiently.

The term “new skin environment” as used herein, denotes a skin region created by the ablation of the stratum corneum and formation of a plurality of micro-channels, using the apparatus of the present invention.

According to the invention, the apparatus for enhancing transdermal delivery of an oligosaccharide comprises: an electrode cartridge, optionally removable, comprising a plurality of electrodes, and a main unit comprising a control unit. The main unit loaded with the electrode cartridge is also denoted herein ViaDerm (see Sintov et al. ibid, the content of which is incorporated by reference as if fully set forth herein).

The control unit is adapted to apply electrical energy of radio frequency (RF) to the electrode typically by generating current flow or one or more sparks when the electrode cartridge is in vicinity of or in contact with the stratum corneum of the skin. The electrical energy applied to the electrodes causes resistive heating and subsequent ablation of stratum corneum in an area beneath the electrodes, thereby generating a plurality of micro-channels. Radio frequency (RF) is meant to refer to a field having a frequency between about 10 kHz and 4000 kHz, preferably between about 10 kHz and 500 kHz.

The control unit comprises circuitry which enables to control the magnitude, frequency, and/or duration of the electrical energy delivered to the electrodes, in order to control current flow or spark generation, and consequently to control the dimensions and shape of the resulting micro-channels. Typically, the electrode cartridge is discarded after one use, and as such is designed for easy attachment to the main unit and subsequent detachment from the unit.

To minimize the chance of contamination of the cartridge and its associated electrodes, attachment and detachment of the cartridge is performed without the user physically touching the cartridge. Preferably, cartridges are sealed in a sterile cartridge holder, which is opened immediately prior to use, whereupon the main unit is brought in contact with a top surface of the cartridge, so as to engage a mechanism that locks the cartridge to the main unit. A simple means of unlocking and ejecting the cartridge, which does not require the user to touch the cartridge, is also provided.

Additional embodiments of the present invention incorporate methods and apparatus described in U.S. Pat. Nos. 6,148,232 and 6,611,706, which are incorporated by reference as if set forth herein. U.S. Pat. No. 6,148,232 to Avrahami discloses an apparatus for applying electrodes at respective points on skin of a subject and applying electrical energy between two or more of the electrodes to cause resistive heating and subsequent ablation of the stratum corneum primarily in an area intermediate the respective points. Various techniques for limiting ablation to the stratum corneum are described, including spacing of the electrodes and monitoring the electrical resistance of skin between adjacent electrodes. The Device for Transdermal Drug Delivery and Analyte Extraction of the type disclosed in U.S. Pat. No. 6,148,232, and various modifications to that invention including those disclosed in U.S. Pat. Nos. 5,983,135, 6,597,946, 6,611,706, 6,708,060, incorporated by reference as if fully set forth herein, are encompassed in the present invention.

U.S. Pat. No. 6,611,706 describes maintaining the ablating electrodes either in contact with the skin or up to a distance of about 500 microns therefrom. Thus, the term “in vicinity” of the skin as used throughout the specification and claims encompasses a distance of 0 to about 500 microns from the electrodes to the skin surface.

Alternatively or additionally, some embodiments of the present invention incorporate methods and apparatus described in the U.S. Pat. No. 6,708,060 entitled “Handheld apparatus and method for transdermal drug delivery and analyte extraction,” which is incorporated by reference as if set forth herein.

Typically, the cartridge supports an array of electrodes, preferably closely-spaced electrodes, the overall area of micro-channels generated in the stratum corneum by the electrode array is small compared to the total area covered by the electrode array.

According to some embodiments, the diameter of the electrodes within an electrode array is in the range of about 30 to about 150 microns. According to certain exemplary embodiments, the diameter of the electrodes within an electrode array is in the range of about 40 to about 100 microns. According to other embodiments, the length of the electrodes is in the range of about 30 to about 500 microns. According to some embodiments, the length of the electrodes is in the range of about 40 to about 150 microns. According to a certain exemplary embodiment, the length of the electrodes is of about 50 microns.

According to additional embodiments, the micro-channels are generated at a density ranging from about 75 micro-channels/cm² of skin to about 450 micro-channels/cm² of skin. Preferably the micro-channels are generated at a density ranging from about 150 micro-channels/cm² to about 300 micro-channels/cm² of skin.

### Uses of the Transdermal System

The present invention further provides a method for transdermal delivery of an oligosaccharide using a transdermal delivery system according to the principles of the present invention. Typically, the procedure for forming new skin environment comprises the step of placing over the skin the apparatus for generating a plurality of micro-channels. Preferably, prior to generating the micro-channels, the treatment sites will be swabbed with sterile alcohol pads. More preferably, the site should be allowed to dry before treatment.

According to certain exemplary embodiments of the present invention, the type of apparatus used to generate micro-channels is particularly disclosed in Sintov et al., ibid, and in WO 2004/039428; as well as in U.S. Pat. Nos. 6,148,232 and 6,708,060; the content of which is incorporated by reference as if fully set forth herein. The apparatus containing the electrode array is placed over the site of treatment, the array is energized by RF energy, and treatment is initiated. In principle, the ablation and generation of micro-channels is completed within seconds. The apparatus is removed after micro-channels are generated at limited depth, preferably limited to the depth of the stratum corneum and the epidermis. A patch according to the principles of the present invention is then attached to the new skin environment.

The present invention provides a method for treating a subject suffering from a thromboembolic disease, the method comprises the following steps:
(i) generating a plurality of micro-channels in an area of the skin of a subject in need of such treatment;
(ii) affixing a skin patch to the area of the skin in which the plurality of micro-channels is present, the patch comprises a pharmaceutical composition comprising a therapeutically effective amount of an oligosaccharide having anti-coagulant activity and a pharmaceutically acceptable carrier; and
(iii) delivering the oligosaccharide into the blood circulation.

According to some embodiments, the oligosaccharide having anti-coagulant activity comprising, in its anionic form, at least the general formula I:

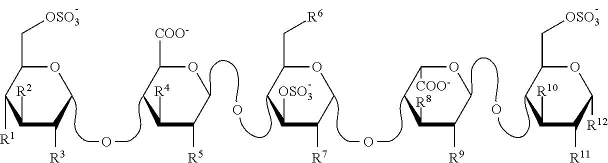

wherein:
R₁ and R₁₂ each is independently OH, (C₁-C₄) alkoxy, or an oligosaccharide consisting of 1-8 monosaccharide units,
R₂, R₄, R₅, and R₈ each is independently OH, (C₁-C₄) alkoxy, or OSO₃⁻,
R₃, R₇, and R₁₁ each is independently NHSO₃ (C₁-C₄) alkoxy, OH, or OSO₃⁻,
R₆ and R₉ each is independently OSO₃⁻, or (C₁-C₄) alkoxy, and
R₁₀ is OH, (C₁-C₄) alkoxy, OSO₃⁻, or COO⁻;
or a pharmaceutically acceptable salt thereof.

The present invention further provides a method for transdermal delivery of an oligosaccharide comprising the steps of:
(i) generating a plurality of micro-channels in an area of the skin of a subject in need of such treatment;
(ii) affixing a patch to the area of the skin in which the plurality of micro-channels is present, the patch comprises a pharmaceutical composition comprising a therapeutically effective amount of an oligosaccharide or a pharmaceutically acceptable salt thereof, and a pharmaceutically acceptable carrier; and
(iii) delivering the oligosaccharide into the blood circulation.

According to some embodiments, the oligosaccharide has anti-coagulant activity. According to additional embodiments, the oligosaccharide having anti-coagulant activity comprising, in anionic form, at least the general formula I.

The term “treating” is meant to include amelioration of the clinical condition of a subject and/or the protection, in whole or in part, against a pathological condition or disease. A “therapeutically effective amount” of the oligosaccharide is that amount of the oligosaccharide which is sufficient to provide a beneficial effect to the subject to whom the oligosaccharide is administered. More specifically, a therapeutically effective amount means an amount of the oligosaccharide effective to prevent, alleviate or ameliorate tissue damage or symptoms of a disease in the subject being treated.

According to some embodiments, the oligosaccharide is a sulfated pentasaccharide. According to certain embodiments, the pentasaccharide is fondaparinux or a pharmaceutically acceptable salt thereof, preferably fondaparinux sodium. According to another embodiment, the pentasaccharide is idraparinux or a pharmaceutically acceptable salt thereof, preferably idraparinux sodium.

The amount of the oligosaccharide to be administered, the duration of treatment or duration of exposure to the oligosaccharide will be determined by the clinician taking into consideration the disease to be treated, the clinical state of the subject, as well as secondary factors including the gender, age, and general physical condition of the patient.

The oligosaccharides of the present invention are useful for treating and preventing thrombin-mediated and thrombin-associated diseases. Thrombin-mediated and thrombin-associated diseases include thrombotic and prothrombotic states in which the coagulation cascade is activated. The present invention thus provides methods for treating venous thromboembolic diseases and arterial thromboembolic diseases which include, but are not limited to, deep vein thrombosis, pulmonary embolism, thrombophlebitis, arterial occlusion from thrombosis or embolism, arterial reocclusion during or after angioplasty or thrombolysis, restenosis following arterial injury or invasive cardiological procedures, postoperative venous thrombosis or embolism, acute or chronic atherosclerosis, stroke, and myocardial infarction. The oligosaccharides of the invention may also be used as inhibitors of smooth muscle cell proliferation, for the treatment of retrovirus infections, like HIV and for the treatment of cancer and neurodegenerative diseases.

According to some embodiments, the methods of the present invention enable delivering of at least about 1 mg oligosaccharide, alternatively of at least about 2 mg, or at least about 5 mg of the oligosaccharide into the blood circulation.

The term “about” refers throughout the specification and claims to an amount of 10% below or above the value, e.g., mg, indicated.

The methods of the present invention provide achieving a therapeutic plasma concentration of the oligosaccharide for an extended period of time. According to some embodiments, the therapeutic plasma concentration of the oligosaccharide such as fondaparinux lasts for at least 10 hours, alternatively for at least 12 hours, 15 hours, 20 hours, or at least 24 hours. As such, the methods of transdermal delivery of the present invention are highly efficacious for obtaining long-lasting effect of the oligosaccharide.

The term “therapeutic” is meant to include amelioration of the clinical condition of a subject and/or the protection, in whole or in part, against a pathological condition or disease.

It is to be understood that the oligosaccharide can be provided alone or in combination with another therapeutic agent. Thus, for example, fondaparinux can be administered transdermally by the methods of the present invention in combination with a compound having anti-platelet aggregation activity (see, for example, U.S. Pat. No. 6,541,488, the content of which is incorporated by reference as if fully set forth herein).

Having now generally described the invention, the same will be more readily understood through reference to the following examples, which are provided by way of illustration and are not intended to be limiting of the present invention.

### Example 1

### Transdermal Delivery of Fondaparinux from a Printed Patch

Transdermal delivery of fondaparinux from a printed patch through micro-channels generated in pig skin by the ViaDerm™ apparatus was evaluated. The experiment included the following groups:
1. Pigs were treated with the ViaDerm™ apparatus to generate micro-channels (MCs) at a density of 150 MCs/cm² and then a printed patch containing fondaparinux (5 mg per 5 cm²) was affixed to the treated skin;
2. Pigs were treated with the ViaDerm™ apparatus to generate micro-channels (MCs) at a density of 300 MCs/cm² and then a printed patch containing fondaparinux (5 mg per 5 cm²) was affixed to the treated skin;
3. Pigs were injected subcutaneously (SC) with 1 mg of fondaparinux.

To prepare the printed patches containing fondaparinux, a concentrated reconstituted solution of Arixtra® (fondaparinux sodium) was applied onto the patch and then air-dried. Each patch was affixed to the ViaDerm™ treated skin for 24 hours. In the control group of SC injection of fondaparinux, Arixtra® (1 mg/0.2 ml) was injected.

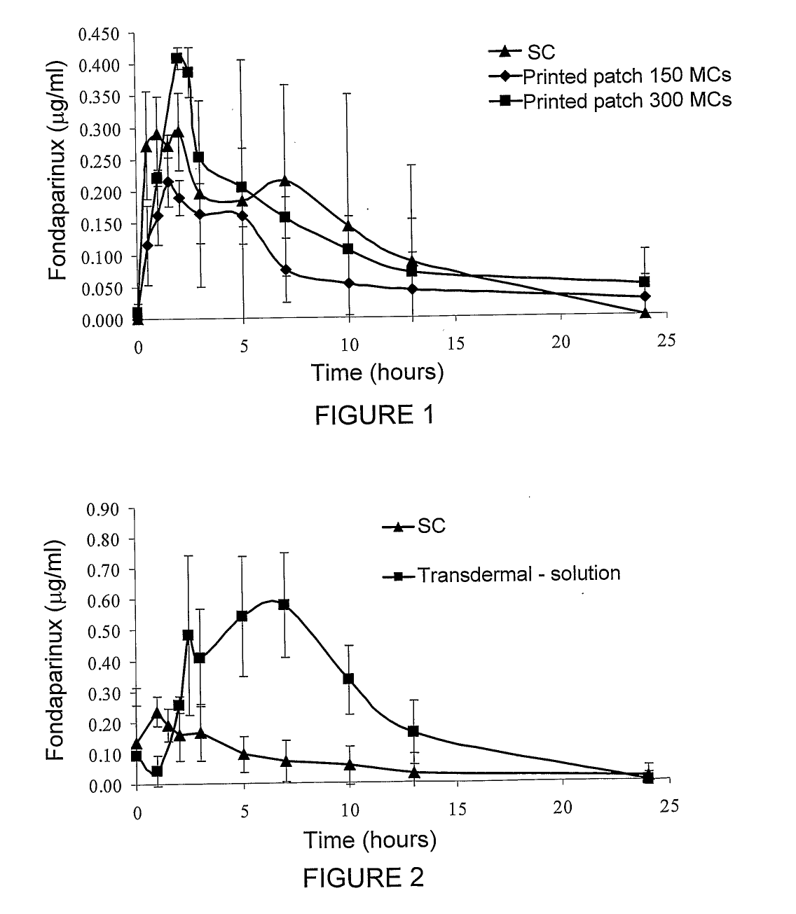

The results of this experiment are shown in FIG. 1 and summarized in Table 1.

TABLE 1
Pharmacokinetic parameters of transdermal delivery
of fondaparinux from printed patches.

| | Cmax* | AUC | | Amount delivered |
|---|---|---|---|---|
| Group | (ng/ml) | (ng*h/ml) | % BA** | (mg) |
| SC 1 mg, n = 4 | 366.17 ± 48.59 | 291 1.25 ± 2242.07 | 100 | |
| TD - 150 MCs | 215.27 ± 38.4 | 2495.5 ± 864.5 | 11.23 ± 1 | 0.56 ± 0.05 |
| PP‡ 5 mg VD | | | | |
| 5 cm², n = 4 | | | | |
| TD - 300 MCs | 417.67 ± 5.37 | 2964.50 ± 1222.59 | 12.74 ± 0.78 | 0.64 ± 0.04 |
| PP 5 mg VD | | | | |
| 5 cm², n = 2 | | | | |

*Cmax average was calculated from individual Cmax for each pig
**BA—Bioavailability
‡PP—printed patch

The results indicated that Tₘₐₓ for SC injection of fondaparinux was observed 2 hours post injection and Cₘₐₓ was 366±48.59 ng/ml. The results for transdermal delivery of fondaparinux from printed patches of 5 mg after generating micro-channels by ViaDerm at a density of 150 and 300 MC/cm² showed Cₘₐₓ of 215±38 ng/ml and Tₘₐₓ after 1.5 hours and Cₘₐₓ of 417.67±5.37 ng/ml, Tₘₐₓ after 2 hours, respectively, of patch application.

The amount of fondaparinux delivered from printed patches was less than 1 mg and the bioavailability was 11-17%. The therapeutic doses of fondaparinux are 2.5-10 mg. Without wishing to be bound to any mechanism of action, the relatively low delivery may be due to high amount of salts in the printed patches. The source of fondaparinux in this study was the commercial drug Arixtra® which contains saline (0.9% NaCl). As the printed patches were prepared following lyophilization of the Arixtra® for injection, 5 mg fondaparinux in each patch contained 3.6 mg of NaCl (42% of the total patch solids). Although the in-vitro dissolution of those patches was high (˜85%), it is likely that the dissolution in the skin was hampered because of the high amount of NaCl resulting in relatively low skin transport. Alternatively, as relatively high volume of an aqueous solution is required to dissolve sugars, and as the volume of exudates released from the micro-channels is small, this volume might not be sufficient to fully dissolve the pentasaccharide.

### Example 2

### Transdermal Delivery of Fondaparinux from a Solution

Transdermal delivery of fondaparinux from a silicon pouch containing the pentasaccharide in solution through micro-channels generated in pig skin by the ViaDerm™ apparatus was evaluated. The experiment included the following groups:
1. Pigs were treated with the ViaDerm™ apparatus to generate micro-channels (MCs) at a density of 300 MCs/cm² and then a silicone pouch patch containing fondaparinux (40 mg per total active delivery area of 2 cm²) was affixed to the treated skin;
2. Pigs were injected subcutaneously (SC) with 1 mg of fondaparinux. The results of this experiment are shown in FIG. 2 and summarized in Table 2.

TABLE 2
Pharmacokinetic parameters of transdermal delivery of fondaparinux
from patches containing the pentasaccharide in solution.

| | Cmax | AUC | | Amount delivered |
|---|---|---|---|---|
| Group | (ng/ml) | (ng*h/ml) | % BA | (mg) |
| SC 1 mg | 235.72 ± 47.79 | 1533.33 ± 1303.96 | 100 | |
| TD ViaDerm Micro 6000, 300 | 709.74 ± 103.61 | 5659.33 ± 956.94 | 20.66 ± 13.64 | 5.72 ± 3.8 |
| MC/cm2 2Xpouches 100 mg/ml | | | | |
| solution 28 mg total dose 2 cm² | | | | |
| total delivery area | | | | |

The transdermal delivery from fondaparinux solution was relatively very high; The amount delivered was 5.7 mg per 2 cm² with bioavailability of 20%. As can be seen from FIG. 2, the pharmacokinetic profile observed when fondaparinux was transdermally delivered from a solution was wider than the pharmacokinetic profile obtained in the SC injected group.

### Example 3

### Transdermal Delivery of Fondaparinux from a Gel Formulation

This study aimed at assessing the transdermal delivery of fondaparinux from hydroxyethyl cellulose (HEC) aqueous gel in comparison to SC administration.

Transdermal delivery of fondaparinux from a silicon pouch containing the pentasaccharide in HEC gel of different viscosities through micro-channels generated in pig skin by the ViaDerm™ apparatus was evaluated. The experiment included the following groups:
1. Pigs were treated with the ViaDerm™ apparatus to generate micro-channels (MCs) at a density of 300 MCs/cm² (at an area of 1 cm²) and then a silicone pouch (1.44 cm²) containing 100 μl of fondaparinux (200 mg/ml) and 1% HEC was affixed to the treated skin (total dose of fondaparinux 13 mg);
2. Pigs were treated with the ViaDerm™ apparatus to generate micro-channels (MCs) at a density of 300 MCs/cm² (at an area of 1 cm²) and then a silicone pouch (1.44 cm²) containing 100 μl of fondaparinux (200 mg/ml) and 1.5% HEC was affixed to the treated skin (total dose of fondaparinux 14 mg);
3. Pigs were treated with the ViaDerm' apparatus to generate micro-channels (MCs) at a density of 300 MCs/cm² (at an area of 1 cm²) and then a silicone pouch (1.44 cm²) containing 100 μl of fondaparinux (200 mg/ml) and 2% HEC was affixed to the treated skin (total dose of fondaparinux 14 mg);
4. Pigs were injected subcutaneously (SC) with 2.5 mg of fondaparinux.

Trans Epidermal Water Loss (TEWL) measurements were performed. The results are shown in Table 3.

TABLE 3
Average results of ΔTEWL (g/hr*m²)

| Group | | ΔTEWL |
|---|---|---|
| # | Treatment | (AVG ± SD) |
| 1 | 1% HEC 13.9 mg/cm², 300 MCs/cm² | 31.2 ± 11.4 |
| 2 | 1.5% HEC 13.9 mg/cm², 300 MCs/cm² | 37.4 ± 7.9 |
| 3 | 2% HEC 13.9 mg/cm², 300 MCs/cm² | 44.4 ± 5.8 |

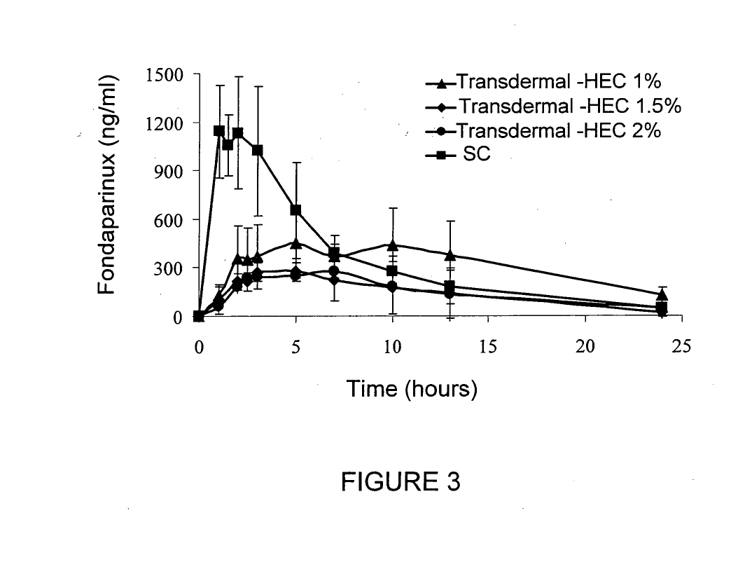

The delivery of fondaparinux (FPX) from pouches containing different viscosities of HEC gel lasted for prolonged period of time as compared to the peak profile obtained by SC injection (FIG. 3). The plasma FPX concentrations obtained after application of the silicone pouch containing HEC gel 1% were maintained at ˜400 ng/ml for 12 hr. These FPX plasma concentrations were higher than those obtained after application of the HEC gel 1.5% and 2% (˜300 ng/ml for 10 hr). The area under curve (AUC) of FPX after application of HEC gel 1% was two fold higher (7491±450) than that obtained after application of HEC gel 1.5% and 2% (3431±2031 and 3549±1925 ng-hr/ml, respectively). The bioavailability (BA) of FPX after application of HEC gel 1% was higher than that obtained after application of 1.5% or 2% HEC gel (15% vs. 7.18% and 7.53%, respectively), and the amount delivered was 2 mg vs. 1 mg and 1.05 mg, respectively.

### Example 4

### Histological Assessment of Skin after Micro-Channel Generation and Treatment with Fondaparinux

Histological assessments were performed in order to determine the effect of application of a high concentration of fondaparinux to skin in which micro-channels have been generated.

Pigs were treated with ViaDerm™ apparatus to generate micro-channels at a density of 450 MCs/cm² (burst length of 6000 μsec). Patches containing fondaparinux at a dose of 1.25 mg (volume of 100 μl of 12.5 mg/ml drug were dried on a backing liner) were applied on the skin and punch biopsies (8 mm diameter puncher) were taken 13 hours and 22.5 hours post application. All biopsies (8 mm diameter) were fixed in 4% formaldehyde buffer for at least 48 hrs followed by staining with eosin and hematoxylin (H&E).

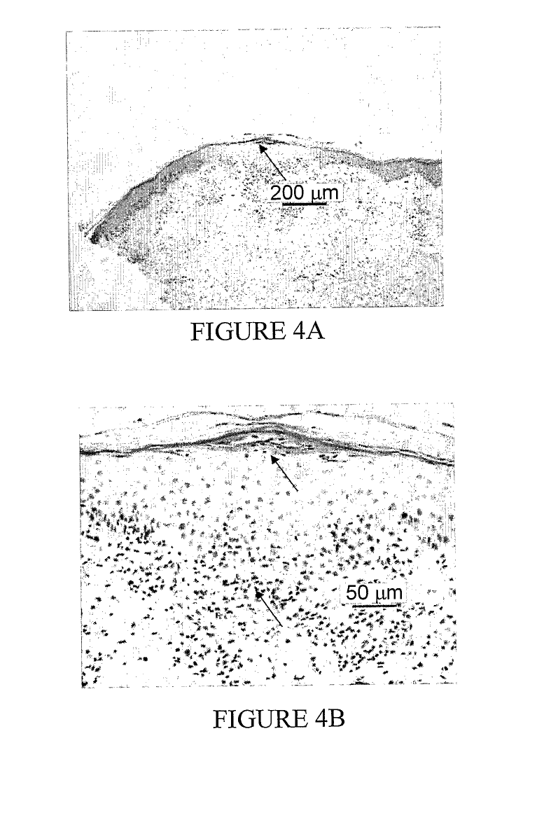

FIGS. 4A-B show that no abnormalities in the skin tissue were observed and no signs of bleeding were detected at the site of the treated skin. The arrows mark the formation of a crust and the recruitment of cells at the application site during the healing process. Thus, transdermal delivery of fondaparinux through micro-channels generated by the apparatus of the present invention enables achieving a therapeutic effect of the pentasaccharide without causing hemorrhage.

It will be appreciated by persons skilled in the art that the present invention is not limited by what has been particularly shown and described herein above. Rather the scope of the invention is defined by the claims that follow.

1.-30. (canceled)

31. A method for treating a subject suffering from a thromboembolic disease, the method comprises:
generating a plurality of micro-channels in an area on the skin of the subject; and
affixing a skin patch to the area of the skin in which the plurality of micro-channels is present, the skin patch comprises a therapeutically effective amount of a pharmaceutical composition comprising as an active agent an oligosaccharide of 5 to 20 monosaccharide units, the oligosaccharide having anti-coagulant activity comprising, in anionic form, at least the general formula: 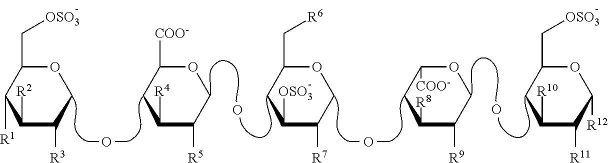
wherein:
R₁ and R₁₂ each is independently OH, (C₁-C₄) alkoxy, or an oligosaccharide consisting of 1 to 8 monosaccharide units,
R₂, R₄, R₅, and R₈ each is independently OH, (C₁-C₄) alkoxy, or OSO₃⁻,
R₃, R₇, and R₁₁ each is independently NHSO₃⁻, (C₁-C₄) alkoxy, OH, or OSO₃,
R₆ and R₉ each is independently OSO₃⁻, or (C₁-C₄) alkoxy, and
R₁₀ is OH, (C₁-C₄) alkoxy, OSO₃⁻, or COO⁻; or a pharmaceutically acceptable salt thereof, further comprising a pharmaceutically acceptable carrier so that the oligosaccharide can be delivered into the blood circulation.

32. The method according to claim 31, wherein the oligosaccharide is fondaparinux or a pharmaceutically acceptable salt thereof.

33. The method according to claim 32, wherein the oligosaccharide is fondaparinux sodium.

34. The method according to claim 31, wherein the oligosaccharide is idraparinux or a pharmaceutically acceptable salt thereof.

35. The method according to claim 31, wherein the pharmaceutical composition is formulated as a viscous liquid, liquid, or in a dry form.

36. The method according to claim 35, wherein the pharmaceutical composition formulated as a viscous liquid further comprising a water-soluble thickening agent.

37. The method according to claim 36, wherein the water-soluble thickening agent is a water-soluble cellulose compound.

38. The method according to claim 37, wherein the water-soluble cellulose compound is present in the composition in an amount ranging from about 0.5% (w/w) to about 3.5% (w/w) of the composition.

39. The method according to claim 35, wherein the viscous liquid has a viscosity of about 300 cps to about 40000 cps.

40. The method according to claim 35, wherein the viscous liquid has a viscosity of about 300 cps to about 3000 cps.

41. The method according to claim 35, wherein the pharmaceutical composition formulated as a viscous liquid comprising fondaparinux sodium, hydroxyethyl cellulose, and water, and wherein the composition having a viscosity of about 300 cps to about 3000 cps.

42. The method according to claim 31, wherein the patch further comprises at least one of a backing layer, an adhesive, a rate-controlling layer, or a release liner.

43. The method according to claim 31, wherein generating the plurality of micro-channels is performed by an apparatus which comprises:
an electrode cartridge comprising a plurality of electrodes; and
a main unit comprising a control unit, which is adapted to apply electrical energy of radio frequency to the electrodes when the electrodes are in vicinity of or in contact with the stratum corneum of the skin, enabling ablation of the stratum corneum in an area beneath the electrodes, thereby generating said plurality of micro-channels.

44. The method according to claim 31, wherein the thromboembolic disease is selected from the group consisting of venous thromboembolic diseases and arterial thromboembolic diseases.

45. The method according to claim 31, wherein the thromboembolic disease is selected from the group consisting of deep vein thrombosis, pulmonary embolism, thrombophlebitis, arterial occlusion from thrombosis or embolism, arterial reocclusion during or after angioplasty or thrombolysis, restenosis following arterial injury or invasive cardiological procedures, postoperative venous thrombosis or embolism, acute or chronic atherosclerosis, stroke, and myocardial infarction.
